(19) United States
(12) Patent Application Publication
(10) Pub. No.: US 2012/0277559 A1
(43) Pub. Date: Nov. 1, 2012

(54) Apparatus for Measuring Blood Parameters

(76) Inventors: Matthias Kohl-Bareis, Sinzig (DE); Branislav Ebert, Burgbrohl (DE); Jens P. Dreier, Berlin (DE); Christoph Leithner, Berlin (DE); Ute Lindauer, Windach (DE); Georg Royl, Berlin (DE)

(21) Appl. No.: 13/514,376

(22) PCT Filed: Dec. 8, 2010
(86) PCT No.: PCT/GB2010/052045

§ 371 Date: Jul. 17, 2012

(30) Foreign Application Priority Data
Dec. 8, 2009 (GB) 0921477.6

(51) Int. Cl. A61B 5/0265; A61B 6/00; A61B 5/1455

(52) U.S. Cl. 600/324; 600/310; 600/317; 600/322; 600/479

(57) ABSTRACT

Apparatus for measuring blood parameters such as chromophore, for example haemoglobin, concentration and blood flow detects light scattered from tissue surface (20) with a multispectral detector (24) that is sensitive to light across a range of different wavelengths. Algorithms are described that demonstrate extraction of chromophore information from scattered light occupying two, red and green or blue, or three bands of the visible spectrum. Simultaneous extraction of blood flow information from scattered laser light occupying either the same or a distinct spectral band is also described.

This invention relates to the field of blood imaging and monitoring and to apparatus for the simultaneous imaging or monitoring of haemoglobin concentration and blood flow, particularly in the small superficial blood vessels of body tissue.

Both blood flow and haemoglobin concentrations are useful and reliable indicators of illness, body performance and stress on an organ. Haemoglobin is one of the central components of the body and is of crucial importance to all body functions. Blood flow in the small vessels of the skin performs an essential role in the regulation of the metabolic, haemodynamic and thermal state of an individual. The condition of the microcirculation over both long and short time periods can reflect the general state of health. The degree of blood perfusion in the cutaneous microvascular structure often provides a good indicator of peripheral vascular disease and reduction of blood flow in the microcirculatory blood vessels can often be attributed to cutaneous vascularisation disorders. There are therefore many situations in routine clinical medicine in which measurement of the blood flow is important.

In the prior art, many techniques exist to measure individually either blood flow or haemoglobin concentration and recording their changes in biological tissue. The tissue may be any organ of living humans or animals, for example, skin, brain or muscle. To date however, there is not a single imaging apparatus that is capable of measuring simultaneously both haemoglobin concentration and blood flow. Measurements are either made sequentially or on separate tissue areas, with the consequence that they may not correlate. The tissue status may change with time, or over a spatial area. Simultaneous measurement of both haemoglobin and blood perfusion (flow) is important when transient changes are to be monitored. This is particularly the case during any functional activation where changes might last just a few seconds. For example, during cortical activation of brain tissue there is a well-described change in haemoglobin that is both localised and may be of short duration. Another example is in the body's response to exercise, stress or heat: skin or muscular tissue changes are induced but fade over a short period as the body adapts. In such cases a sequential measurement of haemoglobin and blood flow would provide data of limited value. In addition, some physiologically important parameters, such as the metabolic rate of extraction of oxygen require both haemoglobin and blood flow data.

Haemoglobin concentrations can occur in both oxygenated [oxyHb] and deoxygenated [deoxyHb] form. The absorption spectra of these forms differ, as can be observed by comparing the appearance of oxygenated and deoxygenated blood. Standard techniques to measure or monitor haemoglobin concentrations and its oxygenation exploit this. Pulse oximetry is a convenient and well known example that measures the oxygen saturation in arterial blood from a pulsatile component of reflected light. This present invention however is concerned with measurement of blood oxygenation and flow in the microcirculation. That is, oxygen saturation and flow in the capillaries, associated with nutritional flow, and in the small arteries and veins associated with both nutritional and thermoregulatory flow.

The spectroscopic method of measuring oxygen saturation and haemoglobin concentration in the microcirculation uses the well known extinction coefficient spectra of oxyHb and deoxyHb. That is, wavelength-dependent light attenuation is measured and converted into concentrations. Either changes in the haemoglobin component concentrations or their absolute values can be measured. Absolute quantification of haemoglobin allows the oxygen saturation to be calculated:

$$SO 2 = \frac{[ oxyHb ]}{[ oxyHb ] + [ deoxyHb ]}$$

Concentration measurements taken at sample points over a tissue surface area can be used to construct a two-dimensional image. Multiple images of the area may be taken in successive time periods in order to construct a video image, or other time-dependent data collection. Physiologically meaningful information can be extracted either from the time course of different images and/or from different regions of interest in an image.

As an alternative to imaging, haemoglobin concentration can be monitored by taking a single site (pixel) measurement. Monitoring enables data to be collected more rapidly than for imaging, which in turn permits a more accurate time-resolution of physiological changes. For example, tissue oxygenation during sport or exercise may be assessed by monitoring and, in a different setup, brain monitoring provides a useful tool in babies undergoing cardiac surgery.

US 2007/0024946 describes use of a hyperspectral camera to image haemoglobin concentrations. Such a camera is however costly and operates only at a relatively slow frame rate. If the frame rate is too slow, then problems arise with tissue surface movements or displacements during recording of an image.

Izumi Nishidate et al. in “Visualizing of skin chromophore concentration by use of RGB images”, Optics Letters 33 (19) page 2263-2265, 2008, describe how a relatively inexpensive RGB camera can be used to image haemoglobin concentrations. This paper demonstrates the possibility of using a relatively crude spectroscopic analysis, with attenuation data collected from three (red, green and blue) wavelength bands, to extract a measure of chromophore concentration.

Blood flow in the microcirculation (or blood perfusion) is conventionally measured by observing the scattering of monochromatic and coherent light from blood cells moving in illuminated tissue. Laser light that is incident on tissue, typically the skin surface, is scattered by moving red blood cells and undergoes frequency broadening. Two basic techniques are used to analyse this effect: laser Doppler and speckle contrast. Using the laser Doppler technique, the frequency broadened laser light, together with laser light scattered from static tissue, is detected and the resulting photocurrent processed to provide a measurement of the average frequency shift that correlates with blood flow. The laser speckle technique observes another manifestation of the frequency broadening, a time-varying speckle pattern. The contrast in the pattern is high for low blood-flow areas and low for high blood-flow areas. Mapping the speckle contrast over a surface area enables a two-dimensional image of blood perfusion to be recorded.

The optical path length of light in tissue is wavelength dependent. Accordingly, different wavelengths can be used to provide information on blood flow at different depths below the tissue surface.

European patent publication number EP 949 880 describes a system capable of real-time display of perfusion over an area of tissue.

It is accordingly an object of the present invention to provide an alternative system for simultaneous haemoglobin and blood flow imaging, which is simpler and less costly than known in the prior art. In addition, there is a need for a portable system that can be readily attached to a patient or other person or animal in order to monitor simultaneously haemoglobin concentration and blood flow.

The present invention provides an apparatus for the simultaneous measurement of blood flow and chromophore concentration, the apparatus comprising:

a multispectral light source for illuminating an area of tissue surface;
a laser source for illuminating the area of tissue surface;
a detector system for detecting light scattered from the tissue, the detector system being arranged to produce a first signal output obtained from detected laser light and a second signal output obtained from detected multispectral light; and
signal processing apparatus arranged to extract blood flow information from the first output signal and chromophore concentration from the second output signal; wherein
the detector system includes a multispectral detector sensitive to light from a range of visible wavelengths to generate the second signal, the signal comprising two channels indicative of light in two respective visible wavelength bands, one of which is a red spectral band.

An important feature of this invention is the ability to extract useful information regarding chromophore concentration using a multispectral detector, which is responsive to light across a range of wavelengths. This broadband detector is preferably a red-green-blue (RGB) detector sensitive to light across the visible spectrum. This is in contrast to many prior art systems in which, more costly, optical filters are used to ensure a narrowband detector response. The detectors used in the present invention may, for example, be charge couple device (CCD) or complementary MOSFET (CMOS) detectors, as used in digital cameras and which are accordingly readily and cheaply available. This present invention has no need of narrowband detectors.

The combination of simultaneous blood flow and chromophore concentration measurements taken using broadband detectors is a first novel aspect of this invention.

The light source, supplying the detected signal from which chromophore concentration measurements are made, is similarly multispectral. By multispectral it is meant that the light emitted occupies two or more wavelength bands of the red-green-blue colour spectrum. The source itself could be a white light source, occupying a bandwidth of around 250 nm, blue through to red. Equally however it could comprise separate LEDs each emitting a wavelength spread of 20 nm to 100 nm (broadband) in one of the red-green-blue parts of the spectrum. Alternatively, it may comprise separate laser sources, each now emitting a narrow bandwidth, but again constrained to occupy distinct (red, green or blue) parts of the visible spectrum. All that is required is that multispectral light is scattered from tissue, enabling detection of separate spectral components by the detector system.

The multispectral light source may emit light in two wavelength bands, corresponding with those used by the multispectral detector to generate the second signal. This then allows the laser source to be arranged to emit light at a wavelength outside these two bands. This may be in the third visible band or, for some applications, in a near infrared spectral band. It is a further novel feature of this invention that allows chromophore concentration to be determined from measurements made from light in just two spectral bands. In the prior art, it had been thought that accurate measurement could only be made if data were available across all three spectral bands. Accordingly, in a second aspect the present invention provides apparatus for the measurement of chromophore concentration, the apparatus comprising:

a multispectral light source emitting light in two spectral bands, one of which is a red spectral band, for illuminating an area of tissue surface;
a detector system for detecting light scattered from the tissue, the detector system being arranged to produce a signal output obtained from detected multispectral light; and
signal processing apparatus arranged to extract chromophore concentration from the output signal, the output signal used by the signal processing apparatus containing no information derived from light outside the two spectral bands.

The combination of these two aspects of the invention results in a particularly powerful tool. Restricting the spectral content of light used for chromophore measurement to two spectral bands and the spectral content of the laser light used for blood flow measurement to a separate band allows ready separation at the detector of the signals for processing. This eases processing requirements and increases potential frame rates, as the illumination will not need to be switched between sources, as in the prior art, to avoid the signals mixing.

The multispectral source used in this apparatus may be provided in a number of ways. It may comprise a first light emitting diode (LED) emitting light in the red spectral band and a second LED emitting light in either a blue or a green spectral band. Alternatively, the broadband (typically 20 nm-100 nm) LEDs may be replaced with narrowband laser sources. In another alternative, the source may be a continuous spectrum source used in combination with a filter arranged to block transmission of light in either a blue or a green spectral band. Suitable continuous spectrum sources are: white light LED, fluorescent lamp or incandescent lamp.

Although only two (red and blue or green) wavelength bands are required to provide sufficient information from which to extract chromophore concentration measurements, the third visible band (green or blue) is, of course, also available for use. As stated previously, the laser light that is needed for simultaneous measurement of blood flow and chromophore concentration may advantageously be at a wavelength that occupies the third band. In another embodiment, information obtained from the third band is used to improve the accuracy of the chromophore measurement, in which case, the multispectral source may comprise first, second and third light emitting diodes (LEDs) emitting light in, respectively, red, blue and green spectral bands. Alternatively, the multispectral source may comprise red, blue and green lasers emitting narrowband light in, respectively, red, blue and green spectral bands. Or the source may be a continuous spectrum source.

In a further advantageous embodiment the signal processing apparatus may additionally used to extract information relating to concentration of a second chromophore from the second output signal. That is, the third band may provide information that, used either alone or in conjunction with information obtained from another band, allows calculation of the concentration of a second, naturally occurring, chromophore species.

Alternatively, the additional available data may be used to extract information relating to concentration of an injected dye. Dyes are commonly used in medical treatment to track flow of a substance through part of the body. Being able to image dye concentrations along with, for example, haemoglobin concentrations and blood flow, offers a powerful aid to diagnosis and/or to understanding of body performance.

Although, in many embodiments, the laser light and multispectral light occupy different bands, this may not always be the case. If they do occupy the same bands, then illumination will need to be switched between sources in order to avoid corruption of one data signal with another. Switching may also be optionally implemented with band separation.

The present invention can be implemented in both imaging and monitoring embodiments. In an imaging embodiment, the multispectral detector preferably comprises a 2-dimensional array of detector elements and the signal processing apparatus is arranged to analyse signals obtained from at least two channels of each detector element and so to obtain information regarding chromophore concentration at sample points relating to each detector element and to output said information to an imaging apparatus arranged to display an image of chromophore concentration. Corresponding detector elements may also be used to image blood flow. In monitoring embodiments, the multispectral detector is preferably responsive to provide a single data signal and the signal processing apparatus is arranged to monitor variations in said signal and hence of chromophore concentration.

In the most preferred application of the present invention, the chromophore is oxyhaemoglobin and/or deoxyhaemoglobin. From measurements of haemoglobin concentrations, the signal processing apparatus may be further arranged to extract information relating to blood oxygen saturation of and/or the metabolic rate of oxygen within the illuminated tissue.

In other embodiments of this invention, blood flow information may be extracted from infrared illumination of tissue. In such embodiments, the detector system should include an IR detector, whose output is used to generate the signal from which blood flow is extracted.

Blood flow information may be extracted from the first (laser) output signal using a variety of known techniques such as laser speckle contrast, speckle temporal variation or a laser Doppler technique.

In a third aspect, the present invention provides a method of simultaneously measuring blood flow and chromophore concentration, the method comprising the steps of:
(a) Illuminating an area of tissue surface with a multispectral light source;
(b) Illuminating the area of tissue surface with a laser light source
(c) Detecting light scattered from the tissue across a range of wavelengths, including that of the laser and at least two component bands of the multispectral source;
(d) Calculating blood flow information from scattered laser light; and
(e) Calculating chromophore concentration from scattered light from at least two component bands of the multispectral source.

Steps (a) and (b) may be carried out concurrently, or alternately (switched).

Embodiments of the invention will now be described by way of example only and with reference to the accompanying drawings.

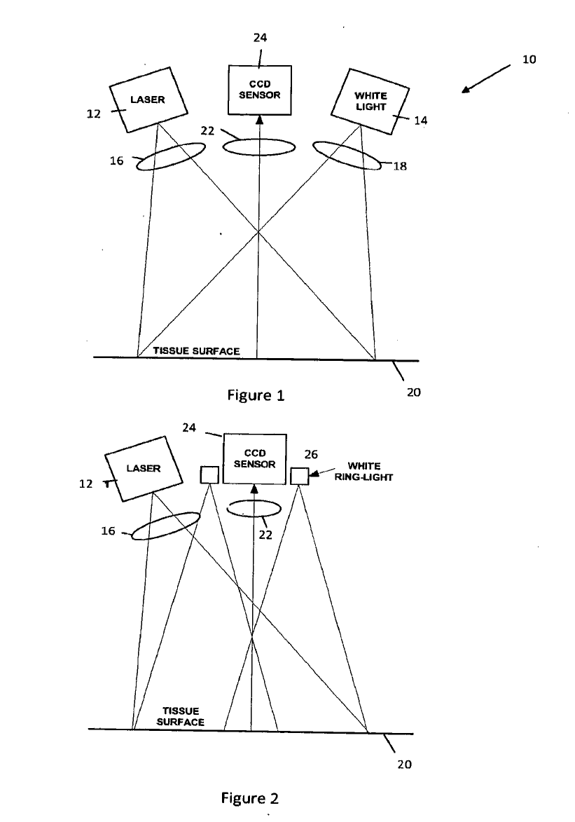

FIG. 1 is a schematic illustration of a first embodiment of a system for imaging blood flow and haemoglobin concentration in accordance with the present invention.

FIG. 2 is a schematic illustration of a second embodiment of an imaging system in accordance with this invention.

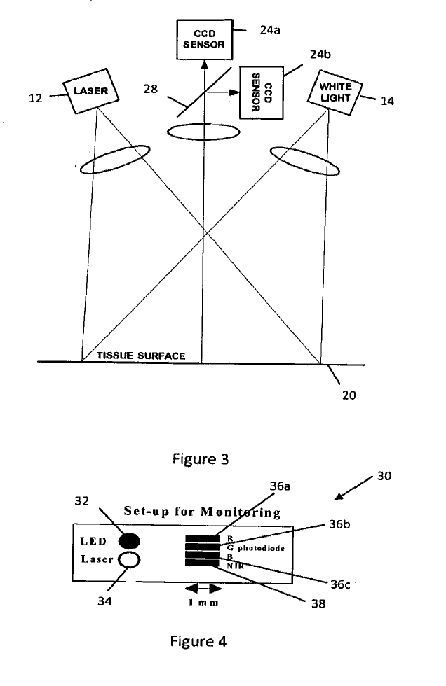

FIG. 3 is a schematic illustration of a third embodiment of an imaging system in accordance with this invention.

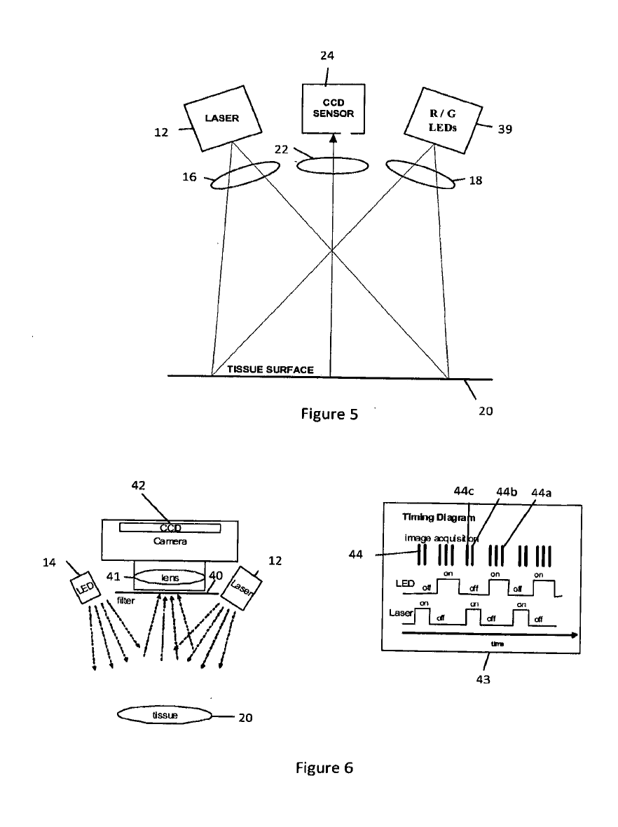

FIG. 4 is a schematic illustration of a layout of a monitor for monitoring blood flow and haemoglobin concentration in accordance with the present invention.

FIG. 5 is a schematic illustration of a fourth embodiment of an imaging system in accordance with this invention.

FIG. 6 is a schematic illustration of a fifth embodiment of an imaging system in accordance with this invention.

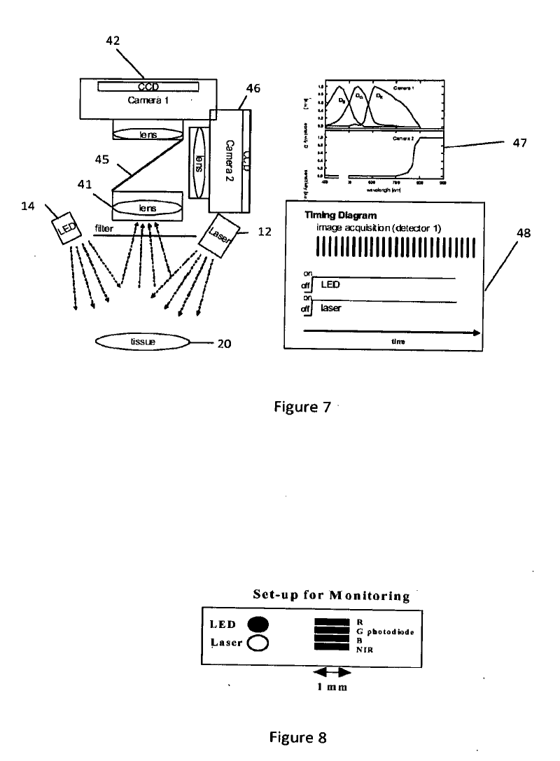

FIG. 7 is a schematic illustration of a sixth embodiment of an imaging system in accordance with this invention.

FIG. 8 is a schematic illustration of a layout of a second embodiment of a monitor for monitoring blood flow and haemoglobin concentration in accordance with the present invention.

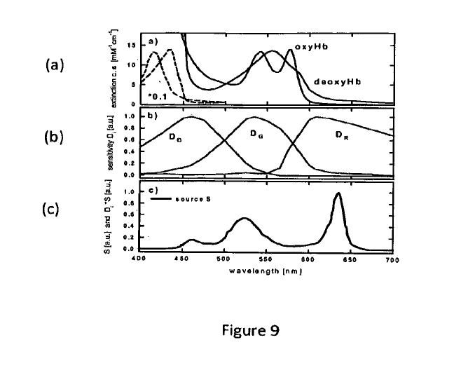

FIG. 9(a) is a graphical plot illustrating the spectral dependence of extinction spectra of oxyHb and deoxyHb over a wavelength range 400 nm to 700 nm.

FIG. 9(b) is a graphical plot illustrating the spectral dependence of sensitivity spectra of D(λ) over a wavelength range 400 nm to 700 nm for blue, green and red elements of a typical RGB detector for use with certain embodiments of this invention.

FIG. 9(c) is a graphical plot illustrating the spectral variation over a wavelength range 400 nm to 700 nm of a typical white light source for use with certain embodiments of this invention.

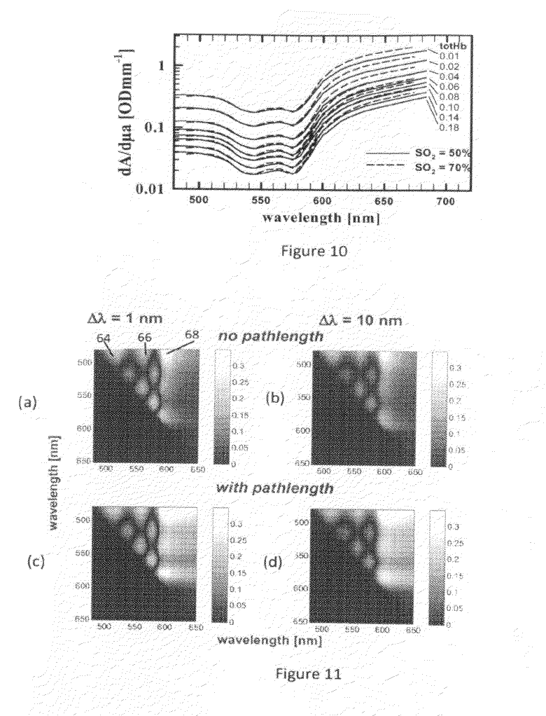

FIG. 10 is a graphical plot illustrating a modelled wavelength dependence of mean photon path length through tissue.

FIGS. 11(a)-(d) are plots showing condition number, an indicator of robustness of a model applied to this invention, for two-wavelength systems, subject to various path length and detection bandwidth assumptions.

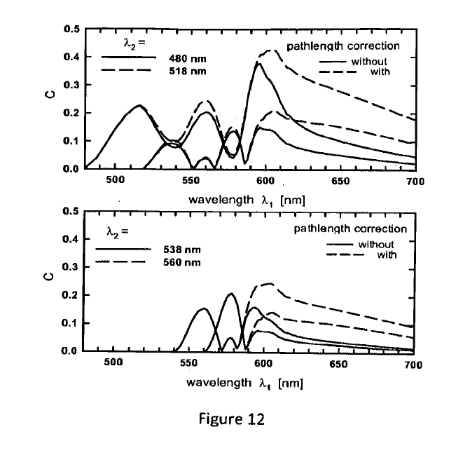

FIG. 12 shows two plots of condition number variation with wavelength of a first wavelength for four selected second wavelength values in a two-wavelength system.

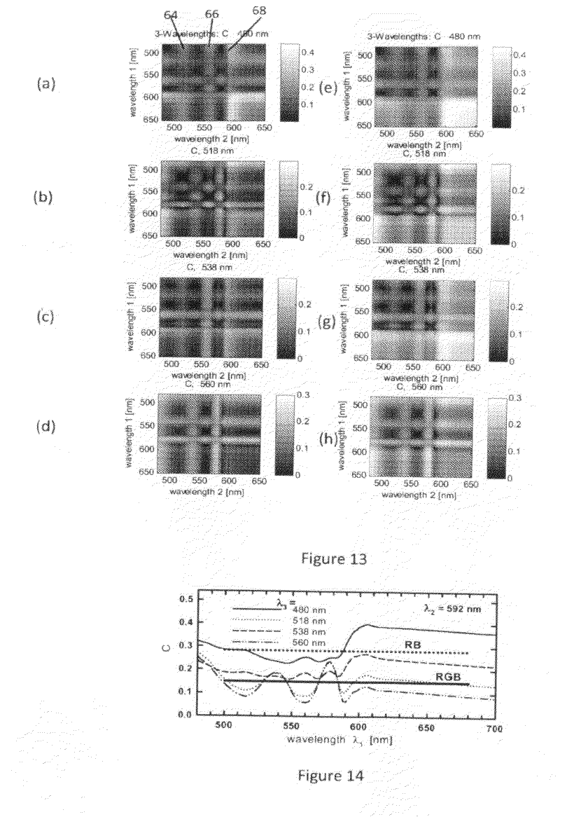

FIGS. 13(a)-(h) are plots showing condition number variation for various three-wavelength systems.

FIG. 14 is a plot showing a cross section of condition number variation with a first wavelength for selected second and third fixed wavelength values in a three-wavelength system.

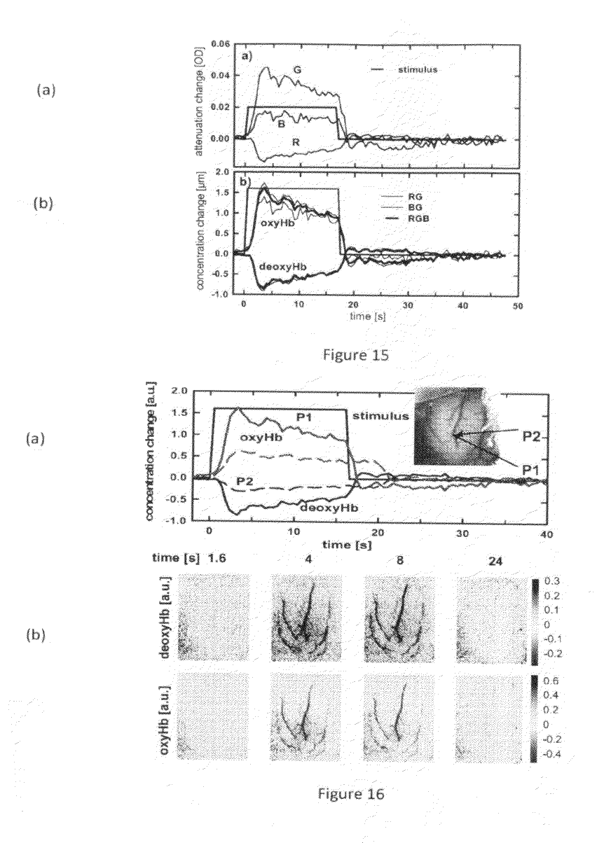

FIG. 15(a) is a plot of measured attenuation change with time in a rat cortex using apparatus in accordance with the present invention, following electrical stimulation (stimulus signal shown overlaid) of a rat forepaw, the plot showing respective measurements for each channel (R,G,B) of an RGB camera detector.

FIG. 15(b) is a graph showing haemoglobin concentrations (for both deoxyHb and oxyHb species) over the same time frame used for FIG. 15(a) the concentrations calculated using either RGB-, RB- or RG-signals shown in FIG. 15(a).

FIG. 16(a) is a graph showing concentration changes of deoxyHb and oxyHb at two spatially separated points (P1, P2) in a rat cortex, following stimulation as for FIG. 15.

FIG. 16(b) is a series of grey scale images of oxyHb and deoxyHb concentrations in a rat cortex following forepaw stimulation, the images being generated using apparatus in accordance with this invention.

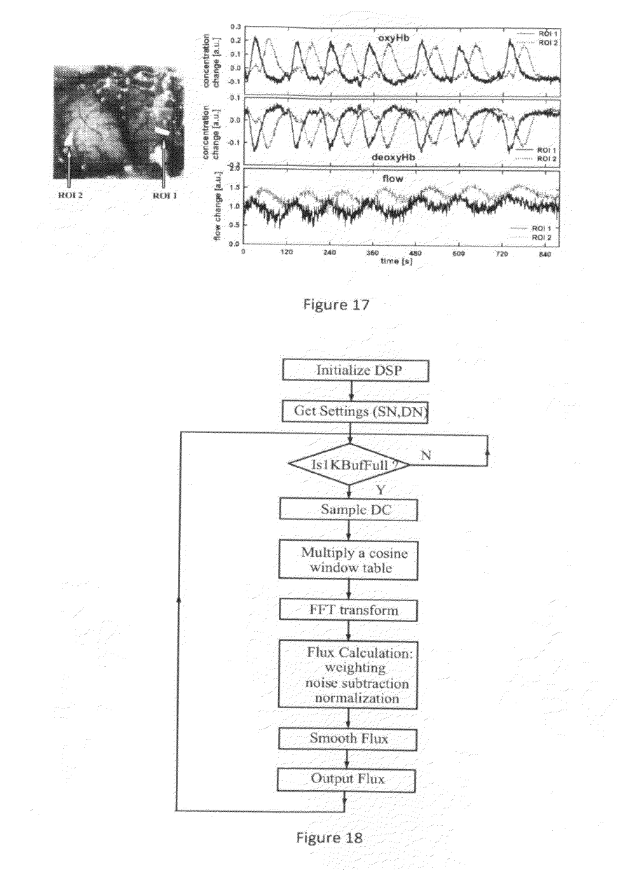

FIG. 17 shows, at its left, an image of a rat cortex indicating two regions of interest (ROI 1 & 2) and, at its right, plots of respectively, oxyHb concentration, deoxyHb concentration and blood flow changes with time as measured at these regions of interest using apparatus in accordance with this invention, following application of a stimulus.

FIG. 18 is a flow chart representing steps involved in implementing an algorithm to extract blood flow data from laser light scattered and reflected from tissue.

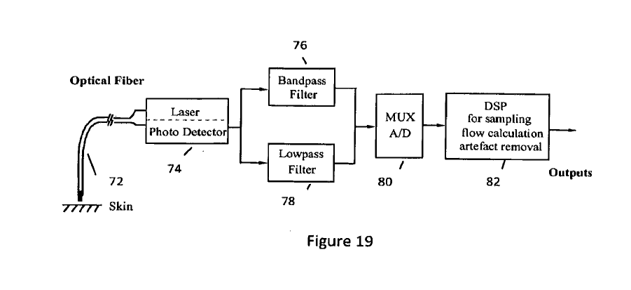

FIG. 19 is a schematic illustration of a system for extracting flux information, suitable for incorporation in an embodiment of the present invention.

With reference to FIG. 1 there is shown a system 10 for imaging blood flow and haemoglobin concentration in accordance with this invention. The system 10 comprises a visible light laser 12 and polychromatic (white) light source 14 whose light is directed by lenses 16, 18 to illuminate a section of tissue surface 20. Light reflected from the surface 20 is collected by a lens 22 and detected by an RGB-CCD (Red, Green, Blue—Charge Coupled Device) detector array 24. The RGB array 24 detects red, green and blue components of light incident on each element of the array. The detected signals are read by a signal processor (not shown) and analysed to extract the required information for each pixel in an image. The analysis process will be described in more detail later. Images are output to a monitor (not shown) for display. The display may be, for example, a false colour image viewed in real time at video frame rates.

Haemoglobin concentrations are extracted by a spectroscopic analysis of light detected from the white light source. Blood flow measurements are extracted from data obtained from a speckle contrast analysis of light detected from the laser source. In taking measurements using this embodiment of the invention therefore, the tissue surface 20 is not illuminated continuously with both light sources 12, 14. The white light source 14 is switched off or blanked while the speckle contrast measurement is made. Similarly, the laser light is prevented from reaching the detector while the haemoglobin measurement is made. This enables data relating to the two measured parameters to be readily separated.

A second embodiment of the invention is illustrated in FIG. 2. In this Figure, the white light source 14 and lens 18 are replaced by a ring lamp LED 26 white light source. In a third embodiment, shown in FIG. 3, two CCD cameras are used to detect the reflected light and the laser 12 is a near infrared laser. A first camera 24a is the RGB-CCD camera detector array as used in the previous embodiment, from which information as to haemoglobin concentration may be derived. A second camera 24b is a near infrared (NIR) detector array, sensitive to a wavelength range that includes that emitted by the NIR laser 12. A dichroic beam splitter 28 directs visible light to the RGB-CCD array 24a and the IR reflected signal to the NIR camera 24b. The signal detected at the NIR camera 24b is used to obtain a speckle-contrast flow measurement.

This embodiment has the advantage that the sensitivity of the speckle contrast flow measurement is significantly improved in comparison with the measurement taken with visible light. Moreover the use of separate detectors means that there is no need to interrupt the white light illumination; the tissue surface can be continuously illuminated by both the white light and NIR laser source. This enables more rapid data collection and so offers the potential for a faster frame rate. Camera frame rate is very important to haemoglobin concentration and blood flow imaging. Blood flow is inherently time-changing and, as mentioned previously, both flow and haemoglobin concentration can change over a short timescale. Imaging at a higher frame rate enables more accurate variations with time to be extracted.

In alternative embodiments, a laser 12 of alternative, for example near-visible, wavelength is used. In this case, the dichroic beam splitter 28 separates this near-visible light from that of the LED white light source. Generally, the beam splitter 28 should be such that it separates incident light into two wavelength bands: one band including the wavelengths of the white light source and the other band the wavelength of the laser source.

In the embodiments illustrated in FIGS. 1-3 the detected signal is processed and analysed by the signal processing apparatus in order to extract data relating to blood flow and haemoglobin concentration. Data is collected from each element in the 2D camera array, the signals (RGB and laser) analysed and the results displayed as a 2D image. The calculation is repeated at successive time intervals, and the displayed image updated and the data stored.

Laser speckle contrast measurements can be made in either of two modes: low resolution spatial processing or high resolution temporal processing. Spatial processing involves the analysis of the intensity variation within small groups (typically 5×5) of pixels within a single frame of image data. Temporal processing involves the analysis of the intensity variation of single pixels over a number of frames (typically at least 25) of image data. In general, temporal processing is capable of generating images with high resolution at relatively low speed, whereas spatial processing generates images with reduced resolution at high speed. The speckle contrast measurements made in these embodiments are extracted using spatial processing. That is, a 2D detector is required with resolution higher than displayed in the image. This provides the potential for relatively high frame rate data collection, which is of course beneficial to situations in which simultaneous measurement of haemoglobin and blood flow are made. RGB-CCD cameras of the type used to image the haemoglobin are available that operate at comparable frame rates.

In alternative embodiments of this invention, a balance is made between camera cost and the desire for high frame rates. Even without the requirement for Doppler laser flow measurements, imaging temporal resolution can be improved at a higher frame rate. In place of the 2D camera, a linear detector array may be used for imaging. A linear detector imager (LDI) can be operated at faster frame rates for considerably less cost than a 2D camera. Consequently, it may find application in many situations.

FIG. 4 illustrates the components of the invention integrated into a small, portable monitor 30. The monitor 30 comprises a white light LED source 32, a single-mode NIR laser diode 34, separate RGB photodiodes 36a, 36b, 36c and a NIR sensitive photodetector 38. In contrast to the imaging embodiments of this invention shown in FIGS. 1 to 3, this embodiment is intended for monitoring only and, as such, uses only point detection. In this embodiment a single photodetector element per channel is used, as opposed to the linear or 2D arrays. As can be seen from the scale included with this figure, these detector elements are ˜1 mm long. The signals detected from the white light source 32 by the RGB photodiodes 36a, 36b, 36c are used to extract haemoglobin concentration measurements. The signal detected from the laser diode 34 at the NIR photodetector 38 is, again in contrast to the imaging embodiments described above, used to extract laser Doppler blood flow measurements. A switching mechanism (not shown) may be included to switch illumination between white visible light and NIR. Alternatively, filters (not shown) may be placed over the RGB detectors 36a, 36b, 36c to remove NIR light and over the NIR detector 38 to remove visible light. Continuous illumination may then be used. In a further alternative, the NIR laser diode 34 is replaced by a visible laser diode and the NIR detector removed. Switching is again implemented between illumination modes and the RGB detectors 36a, 36b, 36c used to detect both the white light and monochromatic laser light, in alternate cycles. The white light detection system can be three separate R, G and B detectors, as shown, or a single RGB silicon diode detector.

The monitor as described with reference to FIG. 4 is small and compact and, ideally, suitable for attachment to a patient or animal, with minimal inconvenience.

The monitor described above uses a point detector that is a compact arrangement of single photodetector elements for each of the wavelength bands used. Alternative monitors may use different techniques to extract a point measurement: for example a single pixel region may be used from a 2D detector array, or a region of interest may be defined by a block of pixels on a 2D array, and the signals detected over the area of the block averaged to obtain a single measurement.

The imaging embodiments of this invention make use of spatial processing of the speckle contrast image. The monitoring embodiment uses a laser Doppler technique, which is advantageous in that it requires only a single element detector. Moreover it can be implemented with direct skin illumination from the laser, via a lens or via a fibre optic cable and direct light collection by the photodetector, via a lens or via an optical fibre. The photodiode can accordingly be very close or in direct contact with the tissue under investigation.

The laser Doppler technique directly detects the frequency spread of scattered light from a Fourier transform of a time-resolved signal. In order to collect sufficient data, a high-frame rate (>5 kHz, ideally) detector must be used. High frame rate 2D imaging detectors are available, but these are not standard and are costly. The output of a single detector element on the other hand can be sampled electronically at suitably high frame rates, which makes the monitor embodiment suitable for implementation with laser Doppler blood flow measurement.

Temporal laser speckle contrast imaging may be used in place of the laser Doppler in the monitor embodiment. This may increase slightly the size of the device as more optical components are required.

The detected signals from the monitor channels are processed and analysed by the signal processing apparatus in order to extract data relating to blood flow and haemoglobin concentration of a small region of tissue surface. The calculation is repeated at successive time intervals, and the measurement accordingly updated.

In both imaging and monitoring embodiments of this invention that are described above, the intensities of the RGB components of detected light, relative to a reference intensity, are sufficient to extract information regarding haemoglobin concentration. That this relatively crude spectroscopic analysis is a viable approach was first demonstrated by Izumi Nishidate et al., referenced above. It has been further discovered by the present inventors however that an even more limited spectral analysis is also, under many circumstances, sufficient to extract the haemoglobin concentrations. Additional embodiments of this invention therefore make use of two visible channels: red and blue or red and green to detect illuminating white light or otherwise multispectral light and one further detector channel: either NIR or the unused visible channel, as befits the laser, to detect the laser light. Use of fewer detectors not only permits the device to be simpler, but also reduces the signal processing requirements.

An embodiment of the invention that uses three visible channels to measure simultaneously haemoglobin concentration and blood flow is shown in FIG. 5. This embodiment differs from that shown in FIG. 1 in that the polychromatic (white) light source is replaced by a source 39 consisting of a pair of LED sources, one of which emits broadband radiation in the red part of the visible spectrum and the other emits broadband radiation in the green part of the spectrum. Light from this dual-band source 39 is directed by lens 18 to illuminate a section of tissue surface 20 The laser source 12 in this embodiment generates a beam of light in the blue part of the visible spectrum. Laser light is directed by lens 16 to illuminate the same area of tissue as that illuminated by the dual-band source. As before, light reflected from the surface 20 is collected by lens 22 and detected by an RGB-CCD (Red, Green, Blue-Charge Coupled Device) detector array 24. The RGB array 24 detects red, green and blue components of light incident on each element of the array. The detected signals are read by a signal processor (not shown) and analysed to extract the required information for each pixel in an image, which is then sent to a display.

Haemoglobin concentrations are extracted by a spectroscopic analysis of light detected from the red-green dual-band light source. Blood flow measurements are extracted from data obtained from a speckle contrast analysis of light detected from the blue laser source. This arrangement enables separation of the two signals at the detector. The RGB array 24 will output three channels per pixel. Data received on the red and green channel is used to derive the haemoglobin concentration; data received on the blue channel is used to derive the blood flow. This arrangement therefore avoids the need for switching, which enables data to be collected continuously relating to both measurement parameters, which in turn offers the potential for a faster imaging frame rate. Better time resolution is therefore available, enabling improved imaging of dynamic events. This arrangement also makes use of a single broadband detector, which may be of a type that is readily and relatively cheaply available.

In an alternative embodiment, the laser source emits in the green part of the visible spectrum and the dual-band LED source emits in both the red and blue spectral bands. Measuring blood flow using green light enables the measurement to be made at a different depth below the tissue surface from that obtained using blue light. Again, the received signals occupy three distinct spectral bands, enabling their ready separation to obtain haemoglobin and blood flow measurements without the need for switching.

In a further embodiment, the dual-band LED source 39 may be replaced with a white light source used in conjunction with a blue light stop band filter. This filter absorbs light in the blue spectral band, with the result that illumination from this source is again dual-band red-green. If the laser source 12 emits blue light, signal separation may again be readily achieved at the detector 24. Alternatively, of course, a green laser source 12 may be utilised with a white light source in conjunction with a green light stop band filter.

In making the haemoglobin concentration measurements, the change in intensity of light scattered from the tissue is measured relative to a reference reading. The reference may be set by a time t₀, it may be a reference phantom with known optical parameters to balance, or it may be the RGB signal of a point (pixel) in the image, which gives a spatial variation of haemoglobin concentration.

It can be shown that the measured attenuation change ΔAᵢ, for each detector element i=Red, Green Blue can be related to the concentration changes Δcᵢ for each chromophore j by a matrix equation:

$$Δ {A}_{i} = {\sum }_{j} Δ {c}_{j} \cdot {E}_{ij}$$

Although this equation, and much of the theory below, applies to any chromophore species, haemoglobin will be used as a specific example both for clarity and because it is the measurement of haemoglobin concentration that is seen as the primary application of this invention. In this case therefore, the index j indicates oxyHb and deoxyHb. The matrix E, with the elements Eᵢⱼ, can be modelled, under certain conditions:

Eᵢⱼ=∫βⱼ(λ)·Dᵢ(λ)·S(λ)·L(λ)·dλ.

where
εⱼ(λ) is the extinction coefficient for each chromophore j
Dᵢ(λ) represent the sensitivity spectrum of each detector element
S(λ) is the normalised intensity spectrum of the light source
L(λ) is the photon mean free path length through the tissue.

As will be explained in more detail below, each of these parameters can either be modelled or obtained empirically and this therefore allows the concentration changes Δcⱼ to be calculated by matrix inversion from observation of attenuation changes.

In fact, it can be shown that the extent of the spectroscopic analysis can be reduced to only two chromatic observations: red with either green or blue. In embodiments that make use of this set up therefore and as shown in FIG. 5, only two detectors or detector elements (RB or RG) need be used to detect signals from which the haemoglobin concentrations are extracted. The third (G or B) may be used to detect the laser signal that provides an indication of blood perfusion when the laser wavelength is adapted to fall within the detection wavelength band. An embodiment utilising a white light source will require switching between sources in order to avoid the white light detected at the third (G or B) detector from corrupting the reading obtained from the laser signal. Alternatively, the white light is filtered or only two LED sources are used, in order that only the laser light contributes to the intensity in this band.

Blood flow measurement is, in accordance with this invention, based on one of two methods: speckle contrast tissue perfusion and laser Doppler blood flow measurements.

In making speckle contrast measurements, the intensity at each pixel (spatial or temporally separated) is measured. The ratio of the standard deviation of each pixel intensity to the mean intensity defines the speckle contrast K. The speckle contrast method assumes that blood perfusion is proportional to the mean velocity v of blood flow. It follows therefore that perfusion is inversely proportional to the correlation time τ_{c} of photons within the tissue. Correlation time τ_{c} may be related to speckle contrast K by the following equation, where T is the integration time of the camera:

$$K = \frac{σ}{〈 I 〉} = {\{ \frac{{τ}_{c}}{2 T} [ 1 - {e}^{( \frac{- 2 T}{τ c} )} ] \}}^{1 / 2} .$$

The correlation time τ_{c} is given by: τ_{c}=1/(akₒv) where:
a is an unknown factor related to the Lorentzian width of the scattered spectrum and the scattering properties of the tissue,
v is the mean velocity and
kₒ is the input light wave number.

The above equation can therefore be used to relate speckle contrast K to tissue perfusion. The speckle contrast can vary between 0 (no speckle, very high perfusion) and 1 (fully developed speckle, very low perfusion).

Other embodiments of the invention make use of the laser Doppler approach to determining blood flow. Reflected and scattered light from moving blood comprises two components: one of which is unchanged in frequency and the other of which has a Doppler broadened frequency due to interactions with moving blood cells in the microvasculature of the tissue. This approach uses digital signal processing to analyse a time-varying intensity signal output from a detector to extract information as to frequency spread. The signal is generally weighted by a multiplier, for example ω, and then Fourier Transformed to produce a measure of the noise subtracted and normalised flux (Fluxₛₙ):
ω weighting:

$${Flux}_{sn} = \frac{{\sum }_{{n}_{1}}^{{n}_{2}} np ( n ) - Noise}{D {C}^{2}}$$

ω² weighting:

$${Flux}_{sn} = \frac{{\sum }_{{n}_{1}}^{{n}_{2}} {n}^{2} p ( n ) - Noise}{D {C}^{2}}$$

Noise=SN×DC+DN

where:
n₁ and n₂ are lower and upper limits of frequency components in the computation,
p(n) is the power spectra density of the nth frequency component,
Noise is the system noise which includes dark noise (DN) and DC proportional shot noise (SN).
DC is a measurement of the intensity of the collected scattered light.

FIGS. 6, 7 and 8 show embodiments of this invention suitable for simultaneous detection of haemoglobin concentrations and blood perfusion extracted from speckle contrast imaging.

With reference to FIG. 6, there is shown an LED white light source 14 and laser 12 arranged to illuminate an area of tissue. Reflected and scattered light passes through a filter 40, lens 41 to a single RGB-CCD camera detector array 42. During the course of image collection, a computer (not shown), the camera 42 or other microprocessing device directs the LED 14 and laser 12 to be switched on and off alternately, and signal data to be collected, in accordance with a prescribed timing pattern. The timing pattern is shown in an offset diagram 43 to the right, with time indicated along a horizontal axis. Data signals are read from the RGB detector at intervals, as indicated by lines 44. It can be seen that data collection occurs when the system is in one of three configurations: white light LED 14 on and laser 12 off 44a; laser 12 on and LED 14 off 44b; and both off 44c. The signal detected when both sources are off provides information as to background noise. The filter 40 suppresses unwanted light from reaching the detector 42. In some embodiments, it may be a polarising filter that blocks specularly reflected light. In others, it may be an absorbing filter to block stray ambient light, or a combination of both.

The data collected during LED illumination and during laser illumination are collected, processed, analysed and stored by a device such as a computer, microcomputer, microcontroller or similar. Signal data are used to construct images indicating blood perfusion and haemoglobin concentrations. The output images are sent for display.

Turning now to FIG. 7 the tissue 20 is again illuminated with the white light source 14 and laser 12. Reflected and scattered light passes through a filter 40, lens 41 to a beam splitter 45. Ideally the beam splitter 45 is dichroic in that it transmits light of one wavelength band, that detected by the RGB camera 42, and reflects light of the other wavelength band to a second camera 46. This second camera 46 is sensitive to light outside the RGB wavelength range (see offset diagram 47), for example to light in the near infrared, which includes the wavelength of the laser source 12. Both cameras 42,46 and associated optics 40, 41, 45 may be integrated in one housing or separate.

In this embodiment, the LED 14 and laser 12 are permanently on during data collection, and the detector signals are sampled at regular time intervals, as shown in the timing diagram 48.

A computer or similar microprocessing unit collects data from both the RGB camera 42 and NIR camera 46. That collected from the RGB camera 42 is processed and analysed to extract information relating to the haemoglobin concentration and that collected from the NIR camera 46 is used to perform speckle imaging in order to extract perfusion data. Both results are imaged, simultaneously or otherwise, under command of a user.

In an alternative embodiment, the beamsplitter 45 is not dichroic and splits both parts of the illuminating spectrum, both detectors therefore receiving light from the entire illuminating range. In this embodiment therefore it is important to ensure that the cameras 42, 46 remain insensitive to wavelengths outside their nominal detection range.

In alternative embodiments, the apparatus may be adapted to take measurements of blood flow using a visible wavelength laser. Timings would then have to be such that the sources are switched on and off alternately, as shown for the embodiment in FIG. 6. Two detectors still provide an advantage over one detector, which could of course be sufficient in a switching system, in that image acquisition is quicker and signal to noise ratio reduced.

In a further alternative, not shown, a currently-available commercial camera may be used or adapted. The commercial camera is equipped with filters and three imaging devices: one to detect each of R, G and B. The addition of a further imaging device for NIR (or other wavelength) speckle detection is feasible.

Such an imaging system may be used with three monochromatic laser sources, Red, Green and Blue, for both haemoglobin concentration measurements and speckle contrast blood flow measurements in the three visible wavelength bands. The addition of a fourth image sensor would allow blood flow measurements at a NIR laser wavelength.

In a further alternative, two of the detectors, either R and B or R and G are sufficient for haemoglobin monitoring. The remaining colour detector (G or B) may therefore be used for speckle imaging when the wavelength of the white light source and the laser are adapted. For example, if haemoglobin detection light sources are used irradiating in the R and B detection bands, the laser wavelength can be chosen to be in the range 530-550 nm, where the crosstalk into the R and B detectors is small (compare with the detector sensitivity plot of FIG. 9(b)). In this embodiment no switching of the white light sources or laser is required.

In FIG. 8 there is illustrated a monitoring device for combined haemoglobin concentration and laser Doppler blood flow measurements.

One form of the monitoring device sensor consists of a white, broadband LED, preferably an SMD (Surface Mount Device), and an RGB-sensor with three separate detectors each of area 1 mm×0.3 mm. The separation of LED and detector is about 3 mm. The sensor signals are amplified and input to a standard PC where variations in the signals are converted into measures of haemoglobin concentration changes. A fourth detector (NIR-sensitivity) is for detecting laser radiation for blood flow monitoring based on the laser Doppler technique.

For measurements of more superficial haemoglobin changes and laser Doppler blood flow the sensors can be of smaller area and positioned with smaller separations between light sources and detectors

The mathematics employed by the signal processing calculations in order to extract haemoglobin concentrations and blood flow data from the detected signals will now be explained. From this it will be clear to one skilled in the art how to program a computer or other standard microprocessor to perform the necessary calculations. Thereafter, further details and embodiments of the invention will be described.

Spectroscopic Method of Haemoglobin Quantification with RGB Detection

The standard approach to the analysis of reflectance spectra is based on the Lambert-Beer equation at each wavelength λ:

$$Δ A ( λ ) = {log}_{10} ( \frac{{R}_{0} ( λ )}{R ( λ )} ) = Δ {μ}_{a} ( λ ) \cdot L ( λ ) = {\sum }_{j} {ɛ}_{j} ( λ ) \cdot Δ {c}_{j} \cdot L ( λ ) \qquad ( 1 )$$

Here the attenuation change ΔA(λ) is calculated from the reflectance intensity R(λ) at time t which is normalized with respect to a reference value R₀(λ)=R(t₀; λ) recorded at reference time t₀. The change in the absorption coefficient,

$$Δ {μ}_{a} ( λ ) = {\sum }_{j} {ɛ}_{j} ( λ ) \cdot Δ {c}_{j} ,$$

is the product of the extinction coefficient εⱼ(λ) and the corresponding concentration change Δcⱼ, with the index j signifying the tissue chromophores. L(λ) is the mean optical path-length in the tissue, which depends on both the scattering and absorption properties and is therefore wavelength dependent.

When both the light source and the detector have broad, overlapping spectra Eq. 1 has to be modified. For an observation with a colour detector the measurement parameter is the intensity integrated over a wavelength range,

Iᵢ=F·Gᵢ·∫R(λ)·Dᵢ(λ)·S(λ)·dλ. (2)

The index i signifies one of the colour sensors red, green or blue (RGB) of the camera (CCD detector). The sensitivity spectra of the detector are represented by Dᵢ(λ) and S(λ) is the normalised intensity spectrum of the light source 14. F is a factor depending on optics, geometry, exposure time and other experimental conditions, which may be held constant throughout the collection of each data set. Gᵢ represents the amplifier gain of the CCD detector.

FIGS. 9(a), 9(b) and 9(c) show the spectral dependence of various parameters of the above equations. FIG. 9(a) shows the extinction spectra of oxyHb 50 and deoxyHb 52 over a wavelength range 400 nm to 700 nm. FIG. 9(b) shows the sensitivity spectra D_{B}(λ), D_{G}(λ), D_{R}(λ) of the blue 54, green 56 and red 58 elements respectively of a typical RGB detector. FIG. 9(c) shows the spectral distribution 60 of a typical white light source S(λ) (LED). The amplifier gain Gᵢ of the CCD detector is set by the control software such that the intensity of the source is observed to be equal in all three colour ranges.

For the conversion of reflectance data into chromophore concentration changes, it is assumed that Eq. 1 is modified by integrating over the broad wavelength ranges covered by each detector:

$$Δ {A}_{i} = {log}_{10} ( \frac{{I}_{0 , i} ( λ )}{{I}_{i} ( λ )} ) = {\sum }_{j} Δ {c}_{j} \cdot \int {ɛ}_{j} ( λ ) \cdot Di ( λ ) \cdot L ( λ ) \cdot S ( λ ) \cdot d λ . \qquad ( 3 )$$

Again, the index i represents R, G or B. The assumption here is that the overlap of the spectra remains constant, and this is fulfilled as the spectra S(λ) and Dᵢ(λ) do not change throughout the data collection as long as the gain Gᵢ is held constant. For Eq. 3 the path-length spectra L(λ) need to be known. It is assumed in some prior art documents that L(λ) has no wavelength dependence. A better model is obtained if, as with this invention, it is estimated for tissue from assumptions of its scattering and absorption properties.

Based on these assumptions, the measured attenuation change can be written as

$$\begin{matrix}Δ {A}_{i} = {\sum }_{j} Δ {c}_{j} \cdot {E}_{ij} \\ with & ( 4 ) \\ {E}_{ij} = \int {ɛ}_{j} ( λ ) \cdot {D}_{i} ( λ ) \cdot S ( λ ) \cdot L ( λ ) \cdot d λ . & ( 5 )\end{matrix}$$

This allows the concentration changes Δcⱼ to be calculated by matrix inversion once the matrix Eᵢⱼ has been determined.

The mean photon path length is obtained from Monte Carlo simulations based on the relationship L(λ)=∂A/∂μₐ and the assumption of a homogeneous geometry of the tissue. Scattering coefficient (μₛ), anisotropy factor (g) and absorption coefficient (μₐ) are chosen to encompass the values found in tissue. The dominant part of the wavelength dependence is due to the haemoglobin absorption which causes the path length to increase markedly for λ>600 nm, i.e. when the absorption is small (see FIG. 9(a)). Details of the dependence of L(λ) on absorption and transport scattering coefficient μₐ and μₛ′ (=μₛ′·(1−g)) can be found in Kohl et al. Physics in Medicine and Biology 45, 3749 (2000). Other models are known and may be used, as will be clear to one skilled in the art. In FIG. 10, which is taken from Kohl et al., the calculated path length is shown as a function of wavelength assuming that the tissue absorption is dominated by haemoglobin of different concentrations (total haemoglobin totHb) and oxygen saturation SO₂.

Based on these modelled and empirically-determined spectra, the 2×3 matrix elements Eᵢⱼ are calculated.

It can thus be seen from this model, that Eq. 4 and 5 provide a simple tool with which to generate maps of haemoglobin changes from reflectance images. Reflectance images may be straightforwardly obtained from intensity measurements relative to that taken at a reference time. The reference is an arbitrary time although it may be selected, for example, as the time at which a stimulus is given to the tissue/patient.

Clearly this model gives a straightforward technique to analyse data obtained from three channels of an RGB detector: multiply by the inverse of matrix E to obtain the change in concentrations of the oxyHb and deoxyHb concentrations. A number of assumptions have been made however in order to derive the Eᵢⱼ matrix and so, before this analysis can be used for diagnostic purposes, it is important to test the robustness of the model. The limitations of this approach are therefore tested below by a crosstalk analysis, an estimate of errors and finally by analysis of real data.

### Crosstalk Analysis: Theoretical Estimation

The main issue is whether attenuation changes detected at different wavelengths or wavelength bands exclude large crosstalk between the haemoglobin components. In a situation with large crosstalk, true (real) changes in the concentration of one haemoglobin component affect the calculation of the other, therefore producing an erroneous result. The extent of crosstalk was estimated first, from the condition number associated with the matrix inversion of the linear system of equations of Eq. 4. For any matrix E (with elements Eᵢⱼ), the condition number is defined as the ratio of its largest singular value to its smallest singular value. It is an estimate of the sensitivity and likely crosstalk when measurements are contaminated by noise or errors in the experimental data. Here C is calculated as the inverse of the condition number (cond) of E,

C=1/cond(E),

with C limited to values between 0 and 1. A value of C close to 1 indicates a well conditioned matrix while a value close to 0 signifies that larger errors and crosstalk are likely.

This theoretical estimate is considered for various experimental systems. First, it is calculated for 2-wavelength and 3-wavelength systems with either a narrow bandwidth of Δλ=1 nm or lower spectral resolution of Δλ=10 nm. Such a situation may arise from a finite bandwidth of, for example, an interference filter. This model gives C-values that can be expected with a standard filter-wheel hyperspectral imaging system and therefore is a yardstick for values obtained with, as required, an RGB—detection system.

In FIG. 11, four plots are shown indicating the C-values obtained from 2-wavelength systems: 2×2 matrices are set up using the extinction values of oxyHb and deoxyHb at two wavelengths (λ₁, λ₂). Each wavelength was stepped from 480 to 650 nm with C plotted in false colour: brighter colours indicating a higher C value. FIGS. 11(a) and 11(b) make no consideration of path length correction, that is L(λ) is assumed to be constant. When considering a narrow detection bandwidth and no path length correction various regions of higher C-values appear indicating a favourable combination of wavelengths. As best wavelengths appear combinations which include λ₁≈480 nm and λ₂≈515 nm (area 64), 560 nm (area 66) or 595 nm (area 68). The third-listed combination 68 appears best with C=0.35. Using other wavelengths like 540 nm, or 560 nm reduces C to values between 0.15-0.20. The plot shown in FIG. 11(b) differs from that shown in FIG. 11(a) in that it includes allowance for a finite bandwidth of Δλ=10 nm, as opposed to the 1 nm assumed for FIG. 11(a). It can be seen that this effect reduces C by up to 20%. For a bandwidth of Δλ=15 nm and 20 nm (data not shown) the reduction of C is up to a further 15%. In all these calculations, wavelengths >610 nm appear to be less advantageous. FIGS. 11(c) and 11(d) show the results of the C calculation from matrices that include allowance for a wavelength-dependence of the mean photon path length. When this factor is included the main effect on C is a significant increase for wavelengths >600 nm. This is further illustrated in FIG. 12 where C is plotted as a function of λ₁ for four selected values of λ₂.

It is apparent from these calculations that the choice of wavelength at which observations are made is crucial to a sensitive detection of haemoglobin.

A similar calculation has also been carried out for a 3-λ-system, which gives C-values that are calculated from a 3×2 matrix. The results are shown in FIGS. 13 and 14.

In FIG. 13, eight plots are shown indicating the C-values obtained from 3-wavelength systems: 3×2 matrices are set up using the extinction values of oxyHb and deoxyHb at three wavelengths (λ₁, λ₂, λ₃). Each wavelength was stepped from 480 to 650 nm with C plotted in false colour: brighter colours indicating a higher C value. FIG. 13 (a) to (d) illustrate maps of C-values as λ₂ and λ₃ are varied between 480 and 650 nm and λ₁ is fixed at 480, 518, 538 and 560 nm, respectively. The effect of path length variation was ignored and the bandwidth fixed at Δλ=1 nm. FIG. 13 (e) to (g) show the same maps but when the effect of path length was included in a calculation of Eᵢⱼ. These plots underline the importance of a careful wavelength selection when using the reduced-spectroscopic analysis provided by RGB detection. Certain combinations of wavelengths provide for well-conditioned matrix behaviour (bright areas in FIGS. 13(a) to (e)); other combinations do not. For better readability of the data shown in FIGS. 13, FIG. 14 illustrates a cross section of C-values obtained when one wavelength is variable (480 nm<λ₁<700 nm) and the other wavelengths are fixed (λ₂=592 nm and λ₃=480 nm, 518 nm, 538 nm or 560 nm). These values are selected to give high values of C. This figure indicates that for a three-wavelength system with narrow band-pass (filter wheel or switched sources) the likely crosstalk strongly depends on the wavelengths used.

These values are the benchmark to compare with the RGB-detection. When including the path length L(λ), a matrix

$${E}_{RGB} = \vert \begin{matrix}0.0263 & 0.0669 & 0.0322 \\ 0.0280 & 0.0614 & 0.0086\end{matrix} \vert$$

was obtained, where the three columns give the extinction values Eᵢⱼ for the blue, green and red detector and the upper and lower row for deoxyHb and oxyHb, respectively. The corresponding C-value is 0.1489 (compared with C=0.0524 without path length term).

The structure of the matrix E_{RGB} offers the possibility that little additional spectroscopic benefit is to be gained from using both the green and blue detectors. The values of the Eᵢⱼ elements for these detectors differ by a factor of about 2.5 (1^{st} and 2^{nd} columns above). Accordingly, the C-value is virtually unaffected when only the red and green values are used (C=0.1489) and in fact increases when only blue and red are used (C=0.282).

These C-values compare favourably with those expected from setups described in the prior art.

i) Dunn et al. (2003) use the wavelengths 560, 570, 580, 590, 600, and 610 nm (bandwidth 10 nm) in a filter wheel setup and this gives:
C=0.185.
   ii) Sakaguchi et al. (2007) used 510, 540, 560 and 580 nm:
C=0.096.
   iii) Hillmann et al. (2007) used 472, 532, 570 and 610 nm:
C=0.203.
   iv) Prakash et al. (2007) used 560, 570, 577, and 610 nm (Δλ=10 nm):
C=0.142.
(Dunn et al. ‘Simultaneous imaging of total cerebral haemoglobin concentration, oxygenation, and blood flow during functional activation’, Optics Letters 28, 1, 2003
Koichiro Sakaguchi, Tomoya Tachibana, Shunsuke Furukawa, Takushige Katsura, Kyoko Yamazaki, Hideo Kawaguchi, Atsushi Maki, and Eiji Okada “Experimental prediction of the wavelength-dependent path-length factor for optical intrinsic signal analysis” APPLIED OPTICS 2007, Vol. 46, No. 14 2769-2777.
Elizabeth M. C. Hillman, Anna Devor, Matthew Bouchard, Andrew K. Dunn, G W Krauss, Jesse Skoch, Brian J. Bacskai, Anders M. Dale, and David A. Boas “Depth-resolved Optical Imaging and Microscopy of Vascular Compartment Dynamics During Somatosensory Stimulation” Neuroimage. 2007 March; 35(1): 89-104
Neal Prakash, Jonathan D. Biag, Sameer A. Sheth, Satoshi Mitsuyama, Jeremy Theriot, Chaithanya Ramachandra, and Arthur W. Toga “Temporal profiles and 2-dimensional oxy-, deoxy-, and total-hemoglobin somatosensory maps in rat versus mouse cortex” Neuroimage. 2007; 37(Suppl 1): S27-S36)

The surprising result of this analysis appears that, in contrast to the currently-held belief that a combination of narrow wavelengths should be used for haemoglobin imaging, the RGB approach is comparable or, in some cases, better than using single discrete wavelengths. This should considerably simplify the equipment needed for haemoglobin concentration monitoring and imaging.

### Experimental Evidence

a) Detection of Haemoglobin with RGB, RG or RB

The cortex of a rat following electrical forepaw stimulation was monitored using an RGB camera. The activation pattern measured from a selected pixel of the RGB camera is shown in FIG. 15. FIG. 15(a) shows the measured attenuation change for each channel of the camera. These values were converted into a measured concentration change for each (oxyHb and deoxyHb) haemoglobin species. For the calculation either RGB-signals, RB-signals or RG-signals were used and, as can be seen from FIG. 15(b), these give comparable haemoglobin changes. FIG. 16(b) shows grey scale images of deoxyHb and oxyHb after cortical activation as observed with an RGB-CCD system. Graphically displayed changes at two selected points on the cortex are also shown in FIG. 16(a).

b) Simultaneous Imaging of Haemoglobin and Blood Flow During Cortical Spreading Depression

During cortical spreading depression (CSD) there is a wave of changes in both blood flow and haemoglobin oxygenation moving at a velocity of a few mm/min over the cortex. This was imaged with a system based on one RGB-CCD and alternating illumination with RGB-LED (for haemoglobin measurement) and laser (λ=780 nm). FIG. 17 shows signals from two regions of interest (ROI 1 and ROI 2) on the cortex of a rat. Undulating changes are observed in oxyHb, deoxyHb and blood flow. There is a clear time lag between the signals from both regions due to the finite speed of blood flow.

c) Calculation of Oxygen Saturation

Oxygen saturation (SO₂) of blood is defined as

$${SO}_{2} = \frac{[ oxyHb ]}{[ oxyHb ] + [ deoxyHb ]} .$$

This equation requires knowledge of the haemoglobin concentrations, but as it is only a relative measure, usually expressed in percent, it is the ratio of oxyHb to deoxyHb that is necessary, rather than absolute concentrations.

There are various published approaches to calculating SO₂ from reflectance spectra, which usually rely on taking a reference measurement. For example, Kohl et al. (2000) demonstrated the measurement of SO₂ in cortical tissue. The extension to the calculation of SO₂ based on the measurement with a RGB-sensor has been shown by Nishidate et al., referenced above.

Clearly therefore, the approach described herein can be used not only to extract haemoglobin concentrations (oxyHb and deoxyHb) but also oxygen saturation of the tissue (SO₂). Accordingly, use of this invention permits oxygen saturation of the tissue to be imaged along with blood flow.

d) Calculation of the Metabolic Rate of Oxygen

It is known that the metabolic rate of oxygen (CMRO₂) can be calculated from both haemoglobin concentration parameters and blood flow (e.g. Mayhew J at al. ‘Increased Oxygen Consumption Following Activation of Brain: Theoretical Footnotes Using Spectroscopic Data from Barrel Cortex’ Neuroimage 13, 975-987, 2001). Following Dunn et al., referenced above, the relative change of CMRO₂ can be obtained from relative values of blood flow CBF (CBFᵣₑₗ) and the relative changes of the total haemoglobin content totHb=oxyHb+deoxyHb (totHbᵣₑₗ) and deoxyHb (deoxyHbᵣₑₗ)

$${CMRO}_{2 , rel} = {CBF}_{rel} \times \frac{{deoxyHb}_{rel}}{{totHb}_{rel}} .$$

The method and apparatus of this invention can accordingly be used to extract data relating to the metabolic rate of oxygen.

### Speckle Contrast Tissue Perfusion Measurement

Tissue perfusion is measured by performing contrast analysis on images acquired from a CCD image sensor. The analysis can be performed using either low resolution spatial processing or high resolution temporal processing. As stated previously, spatial processing involves the analysis of the intensity variation within small groups (typically 5×5) of pixels within a single frame of image data. Temporal processing involves the analysis of the intensity variation of single pixels over a number of frames (typically at least 25) of image data. In general, temporal processing is capable of producing images with high resolution at relatively low speed, whilst spatial processing produces images with reduced resolution at high speed.

The technique is described in the following references:
‘Retinal blood flow visualization by means of laser speckle photography’ J. D. Briers and A. F. Fletcher, February 1982,
Reports, Invest. Ophthalmol. Vis. Sci., Vol 22, No. 2, 255-259.
‘Laser Speckle contrast imaging for measuring blood flow’, J. D. Briers, 2007, Optica Applicata, Vol XXXVII, No. 1-2, 139-152.

Similar image processing algorithms are used for both spatial and temporal approaches to the analysis. For each measurement point in the flux image, the speckle contrast K of a number of pixels in the video image is calculated. For spatial processing this calculation is performed on a square group of pixels in a single frame of image data and for temporal processing the calculation is performed at a single pixel location over a number of frames of image data. Speckle contrast is defined as the ratio of the standard deviation σ to the mean <I> of pixel intensity values within each group:

$$K = \frac{σ}{〈 I 〉}$$

Assuming Brownian motion with Lorentzian power spectrum of the velocity distribution, the relationship between speckle contrast K, correlation time τ_{c}, and camera integration time T can be expressed as:

$$K = \frac{σ}{〈 I 〉} = {\{ \frac{{τ}_{c}}{2 T} [ 1 - {e}^{( \frac{- 2 T}{τ c} )} ] \}}^{1 / 2} \qquad ( 6 )$$

The correlation time τ_{c} is given by: τ_{c}=1/(akₒv) where:
a is an unknown factor related to the Lorentzian width of the
scattered spectrum and the scattering properties of the tissue,
v is the mean velocity and
kₒ is the input light wave number.

If we then assume that perfusion is proportional to the mean velocity v then it follows that it is inversely proportional to the correlation time. Equation (6) can therefore be used to relate speckle contrast K to tissue perfusion. The speckle contrast can vary between 0 (no speckle, very high perfusion) and 1 (fully developed speckle, very low perfusion).

The speckle size at the CCD image sensor is related to the lens magnification M and F-number, F:

Speckle Size≈1.22 (1+M)λF

For best performance the lens aperture is adjusted so that the speckle size is approximately equal to the image sensor pixel size.

### Laser Doppler Blood Flow Theory and Measurements

Laser light that is incident on tissue, typically the skin surface, is scattered by moving red blood cells and undergoes frequency broadening. The frequency broadened laser light, together with laser light scattered from static tissue, is detected and the resulting photocurrent processed to provide a signal that correlates with blood flow. Due to the wavelength-dependence of the optical path length of light in tissue, different wavelengths can be used to provide information on flows at different depths below the tissue surface.

Laser light is directed to the tissue surface either via an optical fibre or as a light beam. For “fibre optic” monitors the optical fibre terminates in an optical probe that can be attached to the tissue surface. One or more light collecting fibres also terminate in the probe head and these fibres transmit a proportion of the scattered light to a photo detector and signal processing electronics. Normal fibre separations in the probe head are a few tenths of a millimetre and consequently blood flow is measured in a tissue volume of typically 1 mm³ or smaller. When a larger volume of tissue is stimulated to vasodilate or vasoconstrict, or where for example a healing process results in increased blood flow, the measured blood flow changes in the small tissue volume is generally taken to be representative of the larger volume.

For laser beam monitors, single point measurements can be made by directing the laser light to the desired point on the surface. The probe in a fibre optic system can be manoeuvred to tissue sites not easily accessible to a laser beam. This enables the fibre optic system to take measurements at these less accessible locations, such as in brain tissue, mouth, gut, colon, muscle and bone.

Perfusion measurements using single and multiple channel fibre optic laser Doppler monitors have been made on practically all tissues and applied in most branches of medicine and physiology. The technique and its application have been described in numerous prior art publications.

It is known that a measurement of perfusion can be extracted from the laser Doppler measurements. This measurement is the first moment of the power spectral density of the photo current produced by the heterodyne mixing of Doppler shifted and unshifted laser light scattered from the microvasculature, commonly referred to as “Flux”.

### Flow Calculation

The following analysis is described in European patent publication number EP 0 949 880.

Laser light reflected and scattered from tissue consists of two components: one of which is unchanged in frequency and the other of which has a Doppler broadened frequency due to interactions with moving blood cells in the microvasculature of the tissue. The performance of any laser Doppler flow monitor (LDF) mainly depends on the nature of the signal processing algorithm and the method of implementing the algorithm. Since the introduction of the first LDF monitor, many different techniques for obtaining a reliable blood flow measurement have been proposed in the prior art. Frequency weighting the detected signal, which essentially introduces a velocity-dependent multiplier into the signal processing, has become the most frequently used method for blood flow monitoring. This algorithm can be expressed as:

ωweighting: Flux=∫_{ω}_{₁}^{ω}^{₂}ωP(ω)dω

Other ω weightings can also be used. For example, an ω² weighting will give increased weight to scattering from fast moving red blood cells:

ω² weighting: Flux=∫_{ω}_{₁}^{ω}^{₂}ω²P(ω)dω

where ω₁ and ω₂ are lower and upper cut-off frequencies of the bandpass filter and P(ω) is the power spectral density.

These algorithms involve the complicated and time consuming computation of a large number of power spectra. Accordingly, most LDFs adopt an analogue approach to implement the above processing. U.S. Pat. No. 4,596,254 however describes a digital processing technique that employs a simplified autocorrelation algorithm in order to achieve continuous and real-time computation of blood flow.

Digital signal processing (DSP) devices can readily perform 1024-point FFT calculations within 10 ms, which makes it possible to compute flow output directly in frequency spectrum form as described in the ω and ω² weighted algorithms. Embodiments of this invention that incorporate laser Doppler flow calculations make use of a DSP device for digital processing of the power spectra of blood flow signals in order to extract a measure blood flow in real-time.

In digital form, the above weighting functions can be written as:

ω weighting: Flux=∫_{ω}_{₁}^{ω}^{₂}ωP(ω)dω=Σₙ_{₁}ⁿ^{₂}np(n)

ω² weighting: Flux=∫_{ω}_{₁}^{ω}^{₂}ωP(ω)dω=Σₙ_{₁}ⁿ^{₂}n²p(n)

and noise subtracted and normalized forms Fluxₛₙ, are
ω weighting:

$${Flux}_{sn} = \frac{{\sum }_{{n}_{1}}^{{n}_{2}} np ( n ) - Noise}{D {C}^{2}}$$

ω² weighting:

$${Flux}_{sn} = \frac{{\sum }_{{n}_{1}}^{{n}_{2}} {n}^{2} p ( n ) - Noise}{D {C}^{2}}$$

Noise=SN×DC+DN

where n₁ and n₂ are lower and upper limits of frequency components in the computation, p(n) is the power spectra density of the nth frequency component, DC is a measurement of the intensity of the collected scattered light, Noise is the system noise which includes dark noise (DN) and DC proportional shot noise (SN).

FIG. 18 shows a flow chart demonstrating implementation of the above algorithms. As an example the Doppler signal (AC) is sampled at 32 kHz and a 1024-point FFT is used. When 1024 points of data have been sampled, data is multiplied by a twiddle cosine window table to reduce artefactual spectral content resulting from discontinuities at the start and end points of the sampled wave form, and is then converted into the frequency domain by FFT. This, along with the weighting function, noise subtraction, normalisation and smoothing are all performed by the DSP. After the FFT transformation of the 1024 points of data is completed, the DSP starts to sample the next 1024 points of Doppler signals in order achieve a higher data rate. The DSP system used in a working embodiment of this invention enables sampling and flux calculation to be performed in approximately 33 ms, resulting in a possible data rate of 30 Hz.

Digital signal processing of the Doppler signal as described above, enables a continuous blood flow output to be produced. It is apparent that both ω and ω² weighting, or other spectral analysis algorithms, can readily be implemented without significantly altering the concept involved. Further, different frequency ranges of the Doppler signal can be analysed separately by suitable selection of the lower and upper limits of frequency components. For example, if it is known that blood flow signal for a particular application is toward a high frequency band, low frequency components can be ignored by increasing the lower limit n₁ to reduce the noise flow output. Another example is to calculate the ratio of flow from a high frequency band to that from a low frequency band using filtered detection. Furthermore, other parameters such as average velocity of the blood flow and red blood cell concentration can be calculated in a similar way.

The average Doppler frequency shift <ω> of the light scattered from moving red blood cells is directly proportional to the average speed of these cells.

$$< ω >= {\int }_{{ω}_{1}}^{{ω}_{2}} \frac{ω P ( ω ) d ω}{{\int }_{{ω}_{1}}^{{ω}_{2}} P ( ω ) d ω}$$

Red blood cell (rbc) concentration is proportional to the integrated power spectral density for low concentration (less than 0.5%) i.e.

rbc concentration ∝∫_{ω}_{₁}^{ω}^{₂}P(ω)dω

FIG. 19 illustrates apparatus suitable for extracting flux information from measurements made on skin. Red or near infra-red light from a low power laser 74 is directed via an optical fibre 72 to skin tissue. Light scattered back from the tissue is collected by one or more other optical fibres 72 and received by a photodetector 74. The photodetector converts the optical signal into an electrical signal. A bandpass filter 76 is used to remove noise outside a selected bandwidth and extract blood flow related AC components. A low-pass filter 78 is also connected to the output of the photodetector 74 and is used to extract DC components, proportional to the intensity of the collected light. Outputs of the bandpass 76 and low-pass filter 78 are converted into digital form by a multiplexer and A/D converter 80. Spectral analysis of the digitised Doppler signal, blood flow calculation and movement artefact detection and removal are performed by a powerful DSP device 82 in real-time.

1. An apparatus for the simultaneous measurement of blood flow and chromophore concentration, the apparatus comprising:
a multispectral light source for illuminating an area of tissue surface;
a laser source for illuminating the area of tissue surface;
a detector system for detecting light scattered from the tissue, the detector system being arranged to produce a first signal output obtained from detected laser light and a second signal output obtained from detected multispectral light; and
signal processing apparatus arranged to extract blood flow information from the first output signal and chromophore concentration from the second output signal; wherein
the detector system includes a multispectral detector sensitive to light from a range of visible wavelengths to generate the second signal, the signal comprising two channels indicative of light in two respective visible wavelength bands, one of which is a red spectral band.

2. Apparatus according to claim 1 wherein the multispectral detector is a red-green-blue (RGB) detector sensitive to light across the visible spectrum.

3. Apparatus according to claim 2 wherein the multispectral detector is selected from one of the following detector types: a charge coupled device (CCD) detector, a complementary MOSFET (CMOS) detector and a silicon photodiode detector.

4. Apparatus according to claim 1 wherein the multispectral light source emits light in two wavelength bands, corresponding with those used by the multispectral detector to generate the second signal and the laser source emits light at a wavelength outside these two bands.

5. Apparatus according to claim 4 wherein the multispectral source comprises a first light emitting diode (LED) emitting light in the red spectral band and a second LED emitting light in either a blue or a green spectral band.

6. Apparatus according to claim 4 wherein the multispectral source comprises a red laser emitting narrowband light in the red spectral band and either a blue laser emitting narrowband light in a blue spectral band or a green laser emitting narrowband light in a green spectral band.

7. Apparatus according to claim 4 wherein the multispectral source comprises a continuous spectrum source and a filter arranged to block transmission of light in either a blue or a green spectral band.

8. Apparatus according to claim 7 wherein the multispectral source is selected from one of the following: white light LED, fluorescent lamp or incandescent lamp.

9. Apparatus according to claim 1 wherein the multispectral detector additionally includes an output channel corresponding to a third visible wavelength band.

10. Apparatus according to claim 9 wherein the first output signal is obtained from the output channel corresponding to the third visible wavelength band.

11. Apparatus according to claim 9 wherein the second output signal is obtained from the three channels of the multispectral detector.

12. Apparatus according to claim 11 wherein the multispectral source emits light in three wavelength bands, corresponding with those detected by the multispectral detector.

13. Apparatus according to claim 12 wherein the multispectral source comprises a first, second and third light emitting diodes (LEDs) emitting light in, respectively, red, blue and green spectral bands.

14. Apparatus according to claim 12 wherein the multispectral source comprises a red, blue and green lasers emitting narrowband light in, respectively, red, blue and green spectral bands.

15. Apparatus according to claim 12 wherein the multispectral source comprises a continuous spectrum source.

16. Apparatus according to claim 11 wherein the signal processing apparatus is additionally used to extract information relating to concentration of a second chromophore from the second output signal.

17. Apparatus according to claim 11 wherein the signal processing apparatus is additionally used to extract information relating to concentration of an injected dye from the second output signal.

18. Apparatus according to claim 9 wherein the apparatus includes a switch arranged to switch illumination between the multispectral source and the laser source.

19. Apparatus according to claim 1 wherein the multi spectral detector comprises a 2-dimensional array of detector elements and the signal processing apparatus is arranged to analyse signals obtained from at least two channels of each detector element and so to obtain information regarding chromophore concentration at sample points relating to each detector element and to output said information to an imaging apparatus arranged to display an image of chromophore concentration.

20. Apparatus according to claim 1 wherein the multispectral detector is responsive to provide a single data signal and the signal processing apparatus is arranged to monitor variations in said signal and hence of chromophore concentration.

21. Apparatus according to claim 1 wherein the chromophore is oxyhaemoglobin and/or deoxyhaemoglobin.

22. Apparatus according to claim 21 wherein the signal processing apparatus is arranged to extract oxyhaemoglobin and deoxyhaemoglobin concentrations from signals obtained at two or three channels of the multispectral detector.

23. Apparatus according to claim 22 wherein the signal processing apparatus is further arranged to extract information relating to blood oxygen saturation of the illuminated tissue.

24. Apparatus according to claim 22 wherein the signal processing apparatus is further arranged to extract information relating to the metabolic rate of oxygen within the illuminated tissue.

25. Apparatus according to claim 1 wherein the laser source emits visible light.

26. Apparatus according to claim 25 wherein the multispectral detector is also responsive to light from the laser source, the output thereof also being used to generate the first output signal from which blood flow is extracted.

27. Apparatus according to claim 1 wherein the laser source emits infrared (IR) light and the detector system includes an IR detector, responsive to light from this laser, the output of the IR detector being used to generate the first output signal from which blood flow is extracted.

28. Apparatus according to claim 1 wherein the signal processing apparatus is arranged to extract blood flow information from the first output signal using laser speckle contrast.

29. Apparatus according to claim 1 wherein the signal processing apparatus is arranged to extract blood flow information from the first output signal using speckle temporal variation.

30. Apparatus according to claim 1 wherein the signal processing apparatus is arranged to extract blood flow information from the first output signal using a laser Doppler technique.

31. Apparatus according to claim 25 wherein the detector system comprises a multispectral detector and a detector sensitive to light from the laser source, each detector comprising a 2-dimensional array of detector elements and the signal processing apparatus is arranged to analyse signals obtained from at least two channels of each multispectral detector element and so to obtain information regarding chromophore concentration at sample points relating to each detector element and to analyse signals obtained from each laser detector element and so to obtain information relating to blood flow at sample points relating to each detector element and to output said information to an imaging apparatus arranged to display an image of chromophore concentration and of blood flow.

32. Apparatus according to claim 25 wherein the detector system comprises:
a multispectral detector and a detector sensitive to light from the laser source, each detector comprising a linear array of detector elements and wherein
the signal processing apparatus is arranged to analyse signals obtained from at least two channels of each multispectral detector element and so to obtain information regarding chromophore concentration at sample points relating to each detector element and to analyse signals obtained from each laser detector element and so to obtain information relating to blood flow at sample points relating to each detector element and
the apparatus further includes imaging apparatus arranged to receive said information from the signal processing apparatus and to display a linear image of chromophore concentration and of blood flow.

33. Apparatus according to claim 32 wherein the detector system includes an optical scanning system arranged to collect successive linear images of illuminated tissue and the imaging apparatus is arranged to display successive linear images as a 2-dimensional display.

34. Apparatus according to claim 32 wherein apparatus includes a scanning system whereby the area of tissue surface is illuminated by the sources as light therefrom is scanned across the surface, scattered light being detected successively from linear sections of the surface, corresponding to the linear detector elements.

35. A method of simultaneously measuring blood flow and chromophore concentration, the method comprising the steps of:
a. Illuminating an area of tissue surface with a multispectral light source;
b. Illuminating the area of tissue surface with a laser source
c. Detecting light scattered from the tissue across a range of wavelengths, including that of the laser and at least two component bands of the multispectral source;
d. Calculating blood flow information from scattered laser light; and
e. Calculating chromophore concentration from scattered light from at least two component bands of the multispectral source.

36. A method according to claim 35 wherein steps (a) and (b) are carried out concurrently.

37. A method according to claim 35 wherein steps (a) and (b) are carried out alternately.

38. Apparatus for the measurement of chromophore concentration, the apparatus comprising:
a multispectral light source emitting light in two spectral bands, one of which is a red spectral band, for illuminating an area of tissue surface;
a detector system for detecting light scattered from the tissue, the detector system being arranged to produce a signal output obtained from detected multispectral light; and
signal processing apparatus arranged to extract chromophore concentration from the output signal, the output signal used by the signal processing apparatus containing no information derived from light outside the two spectral bands.

39. Apparatus according to claim 27 wherein the detector system comprises a multispectral detector and a detector sensitive to light from the laser source, each detector comprising a 2-dimensional array of detector elements and the signal processing apparatus is arranged to analyse signals obtained from at least two channels of each multispectral detector element and so to obtain information regarding chromophore concentration at sample points relating to each detector element and to analyse signals obtained from each laser detector element and so to obtain information relating to blood flow at sample points relating to each detector element and to output said information to an imaging apparatus arranged to display an image of chromophore concentration and of blood flow.

40. Apparatus according to claim 27 wherein the detector system comprises:
a multispectral detector and a detector sensitive to light from the laser source, each detector comprising a linear array of detector elements and wherein
the signal processing apparatus is arranged to analyse signals obtained from at least two channels of each multispectral detector element and so to obtain information regarding chromophore concentration at sample points relating to each detector element and to analyse signals obtained from each laser detector element and so to obtain information relating to blood flow at sample points relating to each detector element and
the apparatus further includes imaging apparatus arranged to receive said information from the signal processing apparatus and to display a linear image of chromophore concentration and of blood flow.

41. Apparatus according to claim 40 wherein the detector system includes an optical scanning system arranged to collect successive linear images of illuminated tissue and the imaging apparatus is arranged to display successive linear images as a 2-dimensional display.

42. Apparatus according to claim 40 wherein apparatus includes a scanning system whereby the area of tissue surface is illuminated by the sources as light therefrom is scanned across the surface, scattered light being detected successively from linear sections of the surface, corresponding to the linear detector elements.
